(19) United States
(12) Patent Application Publication
(10) Pub. No.: US 2026/0354701 A1
(43) Pub. Date: Nov. 12, 2026

(54) Reservoir with Wireless Pressure Monitoring

(71) Applicant: Hangzhou Ruike Medtech Co., Ltd., Hangzhou (CN)

(72) Inventor: Rui XING, Hangzhou (CN)

(21) Appl. No.: 19/719,163

(22) Filed: Jun. 24, 2026

Related U.S. Application Data
(63) Continuation-in-part of application No. PCT/CN2024/143281, filed on Dec. 27, 2024.

(30) Foreign Application Priority Data
Dec. 28, 2023 (CN) 202311833613.9

(51) Int. Cl. A61B 5/03; A61M 27/00

(52) U.S. Cl. CPC A61B 5/031; A61M 27/006

(57) ABSTRACT

A reservoir with wireless pressure monitoring includes a reservoir housing, a ventricular catheter connector, a flexible sealing membrane, and a wireless pressure sensor. The reservoir housing defines a reservoir chamber for storing cerebrospinal fluid and an aspiration/injection window at an upper, scalp-facing portion of the reservoir housing. The ventricular catheter connector is disposed on the reservoir housing and is in fluid communication with the reservoir chamber. The flexible sealing membrane sealingly covers the aspiration/injection window. The wireless pressure sensor includes a sensor body and an antenna, is disposed within the reservoir housing, and is exposed to the reservoir chamber. The wireless pressure sensor is configured to sense a pressure in the reservoir chamber, the pressure being indicative of intracranial pressure (ICP), and to transmit a signal corresponding to the sensed pressure to an external receiver.

### CROSS REFERENCE TO RELATED PATENT APPLICATIONS

This application is continue-in-part of the PCT application with the filing no.

PCT/CN 2024/143281, filed on Dcember 27, 2024, which claims the priority of a prior Chinese application with Application No. 202311833613.9, filed on Dcember 28, 2023. All contents of the mentioned applications are incorporated herein by reference in their entirety.

### FIELD OF THE INVENTION

The present disclosure relates generally to medical devices, and more particularly to a reservoir with wireless pressure monitoring.

### BACKGROUND OF THE INVENTION

Intracranial pressure (ICP) refers to the pressure within the cranium resulting from intracranial contents, including brain tissue, blood, and cerebrospinal fluid (CSF). Under normal conditions, the total volume of these components is maintained in dynamic equilibrium with the intracranial volume, thereby maintaining ICP within a normal range. Severe traumatic brain injury, intracranial hemorrhage, and other acute conditions may cause an acute increase in ICP. Chronic conditions, such as intracranial tumors or hydrocephalus, may also cause increased ICP to varying degrees.

Increased ICP may affect cerebral circulation, resulting in decreased cerebral perfusion pressure, reduced cerebral blood flow, impaired venous return, and intracranial venous congestion. Increased ICP may further cause compression of brain tissue and displacement of brain structures. In severe cases, cerebral herniation may occur, and death may result from secondary brainstem compression or injury. Accordingly, ICP monitoring is commonly used in neurosurgery and neurocritical care to assess ICP and to assist in preventing or managing complications associated with elevated ICP. Intracranial hypertension may be treated, for example, by withdrawing cerebrospinal fluid, administering an osmotic agent such as mannitol, and/or performing decompressive craniectomy.

ICP monitoring may generally be classified as non-invasive monitoring and invasive monitoring. Non-invasive ICP monitoring techniques include, for example, anterior fontanelle pressure measurement, intraocular pressure measurement, transcranial Doppler ultrasound-based assessment of cerebral blood flow, bioelectrical impedance methods, and tympanic membrane displacement tests. Such non-invasive techniques are still under development or clinical evaluation in many applications, and their accuracy and stability may be limited. Invasive ICP monitoring is widely used clinically and may be performed using different approaches depending on whether a pressure sensor is placed intracranially or whether pressure is transmitted through a catheter.

In one invasive approach, a pressure sensor is implanted intracranially through a cranial burr hole or craniotomy and is connected to an external monitor by a lead. This approach may use a relatively small lead diameter, for example less than about 2 mm, and may reduce certain risks associated with larger catheters. However, this approach generally provides pressure monitoring only and does not itself permit CSF withdrawal. If decompression by CSF withdrawal is required, an additional CSF drainage catheter may need to be placed.

In another invasive approach, a catheter is placed into a ventricle, a cistern, or a subarachnoid space, and pressure is measured by a sensor or transducer in fluid communication with CSF in the catheter. This approach allows CSF to be withdrawn or drained while ICP is being monitored, thereby helping to reduce ICP. However, the catheter may have a relatively large diameter, for example greater than about 2.5 mm, and may increase the risk of hemorrhage, tissue injury, and infection. In addition, because the catheter provides a transcutaneous fluid path, the implantation site may remain open or partially open, which may increase infection risk.

In both approaches, a lead or catheter typically passes through the skin. As a result, the implantation site may not be fully closed during monitoring, which may increase infection risk and limit the duration of monitoring. In many clinical settings, wired ICP monitoring is therefore used for a limited period of time, for example about 7 to 10 days. For patients requiring longer-term ICP monitoring, replacement or re-implantation of a sensor or catheter may be required, which may increase healthcare burden and patient discomfort. In addition, a wired monitoring system may limit patient mobility. During patient care, medical staff may need to maintain aseptic handling of the transcutaneous lead or catheter and manage additional complexity introduced by the lead or catheter, thereby increasing the complexity of nursing care.

To reduce one or more of these clinical limitations and to meet the need for longer-term monitoring or out-of-hospital monitoring, wireless ICP monitoring solutions have been proposed. After implantation of a wireless sensor, the scalp may be sutured so that the implantation site is closed. Measurement signals are transmitted through the skin by inductive coupling, thereby reducing infection risk and making such solutions more suitable for longer-term or home monitoring. However, such solutions generally provide ICP monitoring only and do not provide a pathway for withdrawing CSF to reduce ICP.

Certain reservoirs having an ICP monitoring function are also known. In such a solution, a pressure sensor is integrated into a reservoir. The reservoir can be connected to a CSF catheter, provide pressure information for adjustment of a shunt valve, assist in determining whether a ventricular catheter is blocked, and allow CSF to be withdrawn. However, certain existing wireless pressure sensors are based on inductive coupling structures, such as MEMS sensors or NFC sensors. Such sensors typically require coupling coils, which increase implant volume. After integration into a reservoir, the sensor and associated coupling structure may occupy space within the reservoir, thereby affecting CSF withdrawal or reducing the effectiveness of flushing a ventricular catheter.

Further, such wireless pressure sensors based on inductive coupling have limitations such as limited monitoring accuracy, signal drift, low sampling frequency, complex sensor design, and high cost, which may limit clinical adoption and use. In addition, an external receiver based on inductive coupling has a relatively bulky structure because of the coupling coil, as shown in FIG. 1.

### BRIEF SUMMARY OF THE INVENTION

To overcome the conventional disadvantages, the present disclosure provides a reservoir with wireless pressure monitoring.

The reservoir with wireless pressure monitoring includes a reservoir housing, a ventricular catheter connector, a flexible sealing membrane, and a wireless pressure sensor.

In some embodiments, the reservoir housing comprises a reservoir chamber or a chamber for storing a cerebrospinal fluid and an aspiration/injection window at an upper, scalp-facing portion of the reservoir housing. The ventricular catheter connector is disposed on the reservoir housing and is in fluid communication with the reservoir chamber. The flexible sealing membrane sealingly covers the aspiration/injection window. The wireless pressure sensor includes a sensor body and an antenna. The wireless pressure sensor is disposed within the reservoir housing and is partially or wholly exposed to the reservoir chamber or contacted with the chamber. The wireless pressure sensor is configured to sense a pressure inside the reservoir chamber, the pressure being indicative of intracranial pressure (ICP), and to transmit a signal corresponding to the sensed pressure to an external receiver. The wireless pressure sensor is based on a surface acoustic wave (SAW) resonator or a thin-film bulk acoustic resonator (FBAR).

In some embodiments, the reservoir housing is an integral structure; or alternatively, the reservoir housing includes an upper housing and a lower housing that are separately formed and sealingly connected, and the upper housing and the lower housing are connected by bonding or welding.

In some embodiments, the wireless pressure sensor has dimensions less than or equal to 10 mm×3 mm×1 mm. The reservoir housing has a circular cross-section with a maximum diameter of 15 mm to 25 mm and a height of 5 mm to 10 mm.

In some embodiments, the reservoir with wireless pressure monitoring further includes a ventricular catheter. One end of the ventricular catheter is connected to the ventricular catheter connector, and another end of the ventricular catheter is configured to be implanted into an anterior horn of a lateral ventricle or another ventricle of a patient. The ventricular catheter is separately connected to the ventricular catheter connector or is integrally connected therewith.

In some embodiments, the ventricular catheter connector is disposed on a bottom wall or a side wall of the reservoir housing.

In some embodiments, the flexible sealing membrane is a flexible elastic membrane. When the flexible sealing membrane is pressed by an external force, the flexible sealing membrane deforms inwardly toward the reservoir chamber to increase pressure in the reservoir chamber, the increased pressure would force a cerebrospinal fluid in the ventricular catheter to clear or flush a drainage opening of the ventricular catheter.

In some embodiments, the flexible sealing membrane is a planar membrane or a curved membrane having a central portion protruding toward the scalp of a patient.

In some embodiments, the flexible sealing membrane has a substantially uniform thickness; or the flexible sealing membrane includes a needle puncture region located in a central region for a needle to puncture and a deformable region located around an outer periphery of the needle puncture region, and when the flexible sealing membrane is pressed, the deformable region would be deformed. The needle puncture region has a thickness greater than a thickness of the deformable region.

In some embodiments, the flexible sealing membrane is made of natural rubber, silicone, or silicone rubber.

In some embodiments, the reservoir housing is made of a biocompatible polymer. In some embodiments, the reservoir with wireless pressure monitoring further includes a puncture-resistant portion disposed on an inner surface of the reservoir housing, or preferably on the bottom of the reservoir housing. In some embodiments, the puncture-resistant portion is made of a biocompatible metal, a biocompatible ceramic, or a biocompatible polymer having a hardness greater than a hardness of the reservoir housing, so as to prevent a needle from damaging or penetrating a bottom portion of the reservoir housing when the needle is used to puncture the sealing membrane.

In some embodiments, a portion of the reservoir housing at which the sensor body is located or the puncture-resistant portion is made of a biocompatible metal material that could be a part of the antenna.

In some embodiments, a portion of the reservoir housing at which the sensor body is located is made of a biocompatible polymer or a biocompatible ceramic and forms part of an antenna support structure.

In some embodiments, the reservoir housing at a location at which the sensor body is located is made of a biocompatible polymer or a biocompatible ceramic and the reservoir housing can be a part of the antenna.

In some embodiments, the reservoir housing at a location at which the sensor body is located is made of a biocompatible polymer or a biocompatible ceramic, and the antenna is disposed on an inner surface and/or an outer surface of the reservoir housing or the antenna is embedded in the reservoir housing at the location where the sensor body is located.

In some embodiments, when the antenna is disposed on an outer surface of the reservoir housing at the location where the sensor body is located or is embedded in the reservoir housing at the location where the sensor body is located, the reservoir housing has one or more wiring holes, and two ends of the antenna are electrically connected to the sensor body through the one or more wiring holes.

In some embodiments, the antenna has any one of an annular shape, a wavy annular shape, a spiral shape with a uniform line width, a spiral shape with a non-uniform line width, a polygonal patch shape, or a circular patch shape. Alternatively, the antenna includes one or more of a straight line, a bent line, or an arcuate line.

In some embodiments, the sensor body is disposed on an inner side wall or an inner bottom wall of the reservoir housing.

In some embodiments, the sensor body is disposed on the inner bottom wall of the reservoir housing and is located outside a projected area of the flexible sealing membrane; or the sensor body is disposed on the inner bottom wall of the reservoir housing, and the reservoir housing has a protective portion extending to a position below the flexible sealing membrane and overlying the sensor body. The protective portion is configured to block further advancement of a needle toward the sensor body after the needle has pierced through the flexible sealing membrane, thereby protecting the sensor body.

In some embodiments, the reservoir with wireless pressure monitoring further includes a peritoneal/atrial shunt catheter connector disposed on a side wall of the reservoir housing and in fluid communication with the reservoir chamber. In some embodiments, the peritoneal/atrial shunt catheter connector is detachably or selectively connectable to a sealing cap (that could be removalble) or to a peritoneal/atrial shunt catheter connected to a peritoneal cavity or an atrium of a patient. When the peritoneal/atrial shunt catheter connector is not connected to the peritoneal/atrial shunt catheter, the removable sealing cap is detachably connected to the peritoneal/atrial shunt catheter connector to seal the peritoneal/atrial shunt catheter connector.

In some embodiment, the peritoneal/atrial shunt catheter is detachably connected to the peritoneal/atrial shunt catheter connector. The reservoir with wireless pressure monitoring further includes a valve disposed on the peritoneal/atrial shunt catheter connector or on/in the peritoneal/atrial shunt catheter. When the flexible sealing membrane is pressed to flush the ventricular catheter, the valve in a flow path leading to the peritoneal/atrial shunt catheter automatically closes so that cerebrospinal fluid in the reservoir chamber flows toward the ventricular catheter, thereby improving the flushing effect.

In some embodiments, materials of the reservoir housing, the ventricular catheter connector, and the peritoneal/atrial shunt catheter connector are the same or different, and each may be made of a material selected from a biocompatible metal, a biocompatible polymer, or a biocompatible ceramic.

In summary, after the reservoir with wireless pressure monitoring provided by the present disclosure is implanted under the scalp of a patient, the implantation site may be closed by suturing the scalp, thereby interrupting a transcutaneous infection pathway, reducing infection risk, and enabling long-term monitoring. At the same time, the reservoir reduces the risk of safety incidents and data loss caused when a patient, particularly a child, pulls out or breaks a lead of a wired monitoring device. The wireless monitoring function facilitates patient transfer, handling, and free movement.

Further, the sensor technology used in the present disclosure may provide improved accuracy relative to certain existing wired and wireless ICP monitoring devices and may reduce issues relating to accuracy and drift. Compared with wireless products based on LC resonator technology, the present disclosure enables miniaturization of the wireless pressure sensor, supports miniaturized design of the reservoir, and makes the reservoir more suitable for implantation under the scalp. If the reservoir size is kept substantially the same as that of a conventional reservoir, the miniaturized sensor can increase the volume of the reservoir chamber, so that more cerebrospinal fluid is discharged into the ventricular catheter when the flexible sealing membrane is pressed, thereby improving the flushing effect. In addition, the external reader and antenna may also be made smaller, thereby providing higher usability and comfort in use and meeting the need for long-term continuous monitoring of a patient.

The above and other objects, features, and advantages of the present disclosure will become more apparent from the following detailed description of embodiments with reference to the accompanying drawings.

### BRIEF DESCRIPTION OF THE FIGURES

FIG. 1 is a schematic structural diagram of a conventional reservoir equipped with an inductive-coupling pressure sensor when pressure data are being read.

FIG. 2 is a schematic structural diagram of a reservoir with wireless pressure monitoring according to Embodiment 1 of the present disclosure.

FIG. 3 is a schematic diagram showing the reservoir with wireless pressure monitoring of FIG. 2 implanted under the scalp of a patient.

FIG. 4 is a schematic structural diagram of FIG. 2 with the ventricular catheter removed.

FIG. 5 is a schematic structural diagram of FIG. 4 with the flexible sealing membrane removed.

FIG. 6 is a schematic structural diagram of a reservoir with wireless pressure monitoring according to another embodiment of the present disclosure.

FIG. 7 is a schematic diagram showing the reservoir with wireless pressure monitoring of FIG. 6 implanted under the scalp of a patient.

FIG. 8 is a schematic structural diagram of a reservoir with wireless pressure monitoring according to yet another embodiment of the present disclosure.

FIG. 9A is a schematic diagram showing an arrangement of an antenna on an inner bottom surface of the reservoir housing or on a puncture-resistant portion in the reservoir with wireless pressure monitoring of FIG. 2.

FIGS. 9B to 9H are schematic diagrams showing different antenna structures disposed on an inner bottom surface of the reservoir housing or on a puncture-resistant portion of a reservoir with wireless pressure monitoring according to other embodiments of the present disclosure.

FIG. 10 is a schematic structural diagram of a reservoir with wireless pressure monitoring according to one embodiment before being connected to ventricular and peritoneal/atrial shunt catheters.

FIG. 11 is a schematic structural diagram of FIG. 10 after connection to ventricular and peritoneal/atrial shunt catheters.

FIG. 12 is a schematic structural diagram of FIG. 10 when the peritoneal/atrial shunt catheter connector is connected to a removable sealing cap.

FIG. 13 is a schematic structural diagram of a reservoir with wireless pressure monitoring according to yet another embodiment before being connected to ventricular and peritoneal/atrial shunt catheters.

FIG. 14 is a schematic diagram of a packaging structure of a SAW resonator in a sensor body according to an embodiment of the present disclosure.

FIG. 15 is a cross-sectional schematic diagram of the packaging structure shown in FIG. 14.

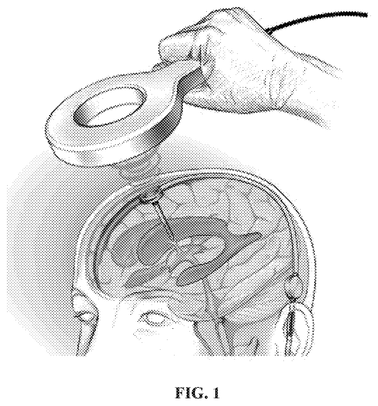

### DETAILED DESCRIPTION

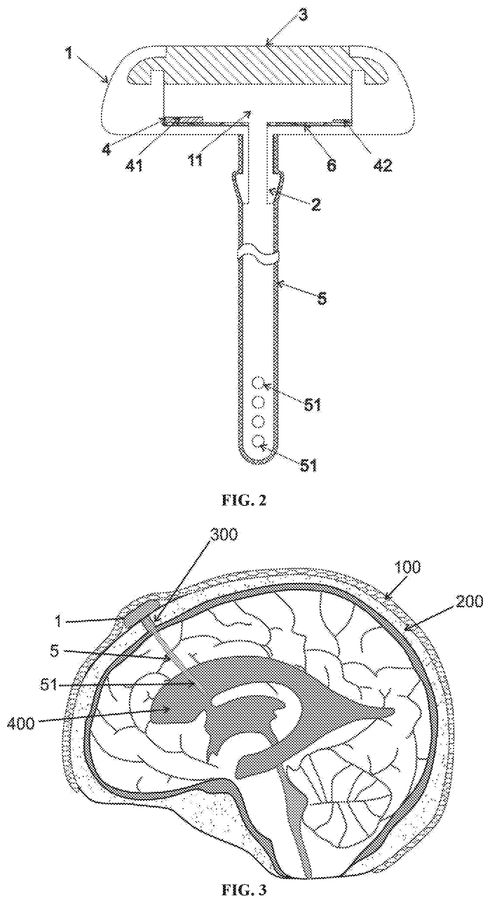

In some embodiments as shown in FIGS. 2 to 5, this invention provides a reservoir with wireless pressure monitoring, including a reservoir housing 1, a ventricular catheter connector 2, a flexible sealing membrane 3, and a wireless pressure sensor 4. The reservoir housing 1 has a reservoir chamber 11 for storing cerebrospinal fluid and an aspiration/injection window or an opening 12, perefered, the window or the opending is at an upper, scalp-facing portion of the reservoir housing 1. In some embodiments, the reservoir chamber 11 has an opening 12 for an aspiration/injection that can be sealed by a membrane 3. The ventricular catheter connector 2 is disposed on the reservoir housing 1 and is in fluid communication with the reservoir chamber 11. The flexible sealing membrane 3 sealingly covers the aspiration/injection window 12, which is also the opening of the reservoir chamber 11. The wireless pressure sensor 4 includes a sensor body 41 and an antenna 42. The wireless pressure sensor 4 is disposed within the reservoir housing 1 or in the reservoir chamber 11, and is partially or wholly exposed or in contact with the reservoir chamber 11, and is configured to sense a pressure in the reservoir chamber 11, the pressure being indicative of intracranial pressure (ICP), and to transmit a signal corresponding to the sensed pressure in the chamber 11 to an external receiver.

Unlike certain existing conventional wireless pressure sensors based on inductive coupling, the reservoir with wireless pressure monitoring provided in this embodiment uses a radio-frequency band, such as an industrial, scientific, and medical band (ISM band), for example 915 MHz or 2.4 GHz. A wireless pressure sensor based on radio-frequency excitation does not require an inductive coil for coupling, thereby reducing the size of the external receiver and the reservoir, so that the reservoir can be miniaturized and implanted under the scalp of a patient where space is limited. After minimally invasive implantation, the scalp may be sutured, thereby reducing risks associated with a transcutaneous infection pathway. Further, because the wireless pressure sensor is excited and powered by radio-frequency energy, the wireless pressure sensor does not require an internal battery or other internal power supply, thereby further reducing the volume of the reservoir.

Specifically, the pressure sensor provided by the present disclosure is a sensor based on a surface acoustic wave (SAW) resonator or a thin-film bulk acoustic resonator (FBAR). Changes in surrounding conditions, such as temperature or pressure, may change the resonant frequency of the sensor and the frequency of an echo signal. The external receiver receives the echo signal transmitted by the wireless pressure sensor and determines pressure based on the frequency of the echo signal. In this embodiment, a pressure sensor based on a SAW resonator or an FBAR is applied to the reservoir in a high-frequency band, such as an industrial, scientific, and medical band (ISM band, for example 915 MHz) or an RFID band, for example 865 MHz. Compared with a conventional LC inductive-coupling pressure sensor having an operating frequency band of 40 MHz to 400 MHz, the high-frequency band does not require a large inductive coil, and therefore the wireless pressure sensor 4 and the external reader can be made smaller. Further, in the high-frequency band, the SAW resonator and the FBAR have a high quality factor Q, typically around 3000, which is much higher than the quality factor of an LC resonator (Q<300).

Specifically, frequency resolution, resonant frequency, quality factor Q, and signal-to-noise ratio SNR have the following relationship:

δf∝f₀/(Q·SNR)

Based on this relationship, a higher quality factor Q results in a smaller frequency resolution δf, that is, higher frequency accuracy. Accordingly, compared with a conventional MEMS pressure sensor based on an LC resonator, the reservoir with wireless pressure monitoring provided in this embodiment can provide improved pressure-sensing sensitivity based on higher frequency resolution, thereby enabling detection of small pressure changes.

At the same time, an increase in the quality factor Q can reduce the resonant bandwidth, thereby reducing noise within the measurement bandwidth and improving the signal-to-noise ratio. Further, a high quality factor Q can provide a sharper and more stable resonant response, enabling improved frequency resolution and reducing measurement uncertainty associated with small frequency shifts. A currently clinically used ICP sensor, such as a MEMS sensor based on an LC resonator, may have an accuracy of about ±2 mmHg. In contrast, the wireless pressure sensor based on a high-Q SAW resonator provided in this embodiment can detect pressure changes of about 1 mmHg. Since ICP values are relatively low, generally ranging from about 7 to 15 mmHg, improved pressure measurement resolution can deliver remarkably beneficial effects for disease diagnosis, therapeutic intervention and disease management.

However, the present disclosure is not limited to any particular type of wireless pressure sensor or any particular manner of transmitting the pressure signal. Other wireless pressure sensors that do not require inductive coupling may also be used. In some embodiments, the reservoir housing 1 is made of a biocompatible metal material, specifically titanium or stainless steel. However, the present disclosure is not limited thereto. In other embodiments, the reservoir housing may also be made of a relatively hard biocompatible polymer, such as polyether ether ketone (PEEK), polyaryletherketone (PAEK), or polytetrafluoroethylene (PTFE), or may be made of a biocompatible ceramic, such as aluminum oxide or zirconium oxide.

In this embodiment, the cross-section of the reservoir housing 1 is circular and has a maximum diameter of 15 mm to 25 mm, and the height of the reservoir housing 1 is 5 mm to 10 mm. Specifically, the size of the reservoir housing 1 may be designed in various specifications according to head sizes of different types of patients, such as adults, children, and infants. Preferably, the reservoir housing 1 may have a maximum cross-sectional diameter of about 15 mm and a height of about 8 mm. However, the present disclosure is not limited thereto. In other embodiments, the cross-section of the reservoir housing may be circular, and the maximum diameter of the cross-section may be another value within 10 mm to 35 mm, and the height of the reservoir housing may also be another value within 4 mm to 15 mm.

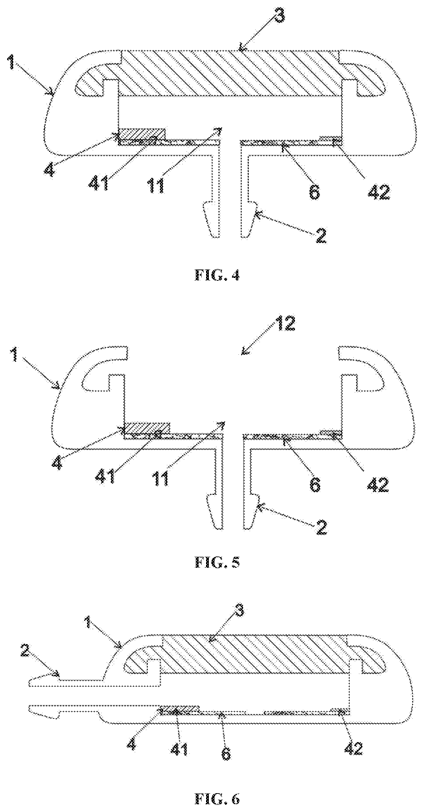

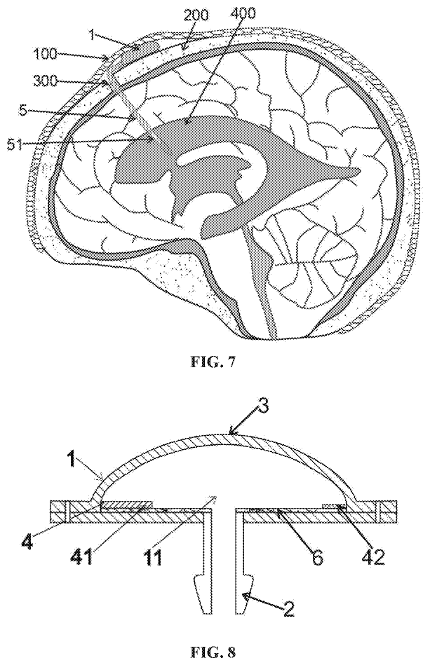

In this embodiment, the reservoir with wireless pressure monitoring further includes a ventricular catheter 5. One end of the ventricular catheter 5 is connected to the ventricular catheter connector 2, and the other end is configured to be implanted into a ventricle of a patient. Specifically, the ventricular catheter connector 2 is disposed on a bottom wall of the reservoir housing 1 and in fluid comunication with the chamber 11, and the ventricular catheter 5 is detachably connected to the ventricular catheter connector 2. FIG. 4 is a schematic structural diagram after the ventricular catheter 5 has been removed. This arrangement allows the reservoir housing 1 and the ventricular catheter connector 2 to be provided or sold as an integrated component. Similarly, the ventricular catheter 5 may also be replaced or sold as an independent component. However, the present disclosure is not limited thereto. In other embodiments, the ventricular catheter 5 may also be integrally connected with the ventricular catheter connector 2. Further, in other embodiments, the ventricular catheter connector 2 may also be disposed on a side wall of the reservoir housing, as shown in FIGS. 6 and 7. In this embodiment, the ventricular catheter 5 could be arcuated or curved to facilitate insertion into a ventricle.

As shown in FIG. 3, the reservoir housing 1 is located between the scalp 100 and the skull 200, preferably on the skull 200 and under the scalp 100. In an exemplary implantation procedure, an incision is made in the scalp 100 to form an implantation site. A cranial burr hole 300 is formed in the skull 200 and typically has a diameter of about 2.7 mm. The ventricular catheter 5 is inserted through the cranial burr hole 300 and advanced into the anterior horn 400 of a lateral ventricle. An excess portion of the ventricular catheter 5 may be trimmed, and the ventricular catheter 5 is then connected to the ventricular catheter connector 2. The reservoir housing 1 is placed above the skull 200 and under the scalp 100. Then, the incision in the scalp 100 is sutured to cover the reservoir housing 1 and close the implantation site, thereby reducing the risk associated with a transcutaneous infection pathway. The anterior horn 400 of the ventricle is in fluid communication with the reservoir chamber 11 through one or more drainage openings 51 of the ventricular catheter 5, so that cerebrospinal fluid can be withdrawn from, and a therapeutic agent can be administered into, the reservoir chamber 11. Specifically, the flexible sealing membrane 3 can provide self-sealing after being pierced by a needle. When the intracranial pressure of the patient is elevated, a needle may be used to puncture the flexible sealing membrane 3 to withdraw cerebrospinal fluid from the reservoir chamber 11, thereby reducing intracranial pressure. In addition, a therapeutic agent may be injected into the reservoir chamber 11 through a needle, and the therapeutic agent may then pass through the ventricular catheter connector 2 and the drainage openings 51 of the ventricular catheter 5 into the ventricle of the patient to provide treatment. In this embodiment, the ventricular catheter connector 2 could be made of the same material as the reservoir housing 1, such as a biocompatible metal material, including titanium or stainless steel. However, the present disclosure is not limited thereto. In other embodiments, the material of the ventricular catheter connector 2 may also be different from that of the reservoir housing and may be a relatively hard biocompatible polymer, such as polypropylene.

In some embodiments, the wireless pressure sensor 4 has dimensions less than or equal to 10 mm×3 mm×1 mm. In the wireless pressure sensor provided by the present disclosure, the antenna 42 is made of a thin biocompatible metal wire or a thin metal sheet. Compared with the volume of the sensor body 41, the volume of the antenna 42 is very small and may be negligible. Therefore, the volume occupied by the wireless pressure sensor 4 may substantially correspond to the volume of the sensor body 41. The small-volume wireless pressure sensor reduces the volume occupied by the sensor in the reservoir chamber 11. Specifically, in the reservoir housing 1 having a circular cross-section with a maximum diameter of 15 mm to 25 mm and a height of 5 mm to 10 mm, the wireless pressure sensor 4 provided in this embodiment occupies less than 5.9% of the internal space of the chamber 11. This space occupancy is much smaller than that of a conventional MEMS sensor in a reservoir housing of the same size, so that the reservoir chamber 11 has a larger fluid storage space.

The relatively large fluid storage space and the elasticity of the flexible sealing membrane 3 for sealing the opening of the chamber 11 allow pressure in the reservoir chamber 11 to be changed. A force may be applied to the flexible sealing membrane 3 on the reservoir housing 1 so that the flexible sealing membrane 3 is depressed toward the reservoir chamber 11, thereby increasing pressure in the reservoir chamber 11. A cerebrospinal fluid in the reservoir chamber 11 and the ventricular catheter 5 is driven toward the drainage openings 51 of the ventricular catheter 5 by the increased pressure in the chamber 11, thereby forceing the cerebrospinal fluid to pass through the drainage openings 51 and reducing or preventing blockage of the ventricular catheter 5, so as to avoid unnecessary catheter replacement surgery. Further, a blockage condition of the ventricular catheter 5 may also be determined or assessed based on a rebound time of the flexible sealing membrane 3 after the membrane is deformed into the chamber 11 by a pressure applied to the membrane 3. As shown in FIG. 2, the ventricular catheter drainage openings 51 are a plurality of circular drainage holes formed at a distal end of the ventricular catheter 5. However, the present disclosure is not limited to any particular shape of the drainage openings of the ventricular catheter, and the drainage openings may have various shapes, such as square, elliptical, or elongated shapes.

Preferably, the ventricular catheter 5 may have an inner diameter of about 1.3 mm and an outer diameter of about 2.5 mm. The length of the ventricular catheter 5 may be trimmed as needed. The ventricular catheter 5 is generally made of a biocompatible polymer, such as silicone, silicone rubber, or natural rubber, and the circular drainage holes 51 are placed in a lateral ventricle for discharging cerebrospinal fluid from the ventricle. However, the present disclosure is not limited thereto.

Specifically, the flexible sealing membrane 3 is a planar flexible elastic membrane, and the flexible sealing membrane may have a substantially uniform overall thickness. A 25-gauge or smaller needle may be used when withdrawing cerebrospinal fluid and injecting a therapeutic agent. However, the present disclosure is not limited thereto. In other embodiments, to allow the flexible sealing membrane to withstand repeated needle punctures and to self-seal after being pierced by a needle, the overall thickness of the flexible sealing membrane may be increased, for example to about 4 mm or greater. However, the present disclosure is not limited thereto. In other embodiments, the flexible sealing membrane may include a needle puncture region located in a middle region and a deformable region located around an outer periphery of the needle puncture region. The deformable region is configured to deform when the flexible sealing membrane is pressed. The needle puncture region has a thickness greater than a thickness of the deformable region. The thicker needle puncture region can increase the number of needle punctures that the flexible sealing membrane can tolerate and extend the service life of the reservoir, while the thinner deformable region facilitates deformation of the flexible sealing membrane when pressed to flush the ventricular catheter 5. In other embodiments, a thinner flexible middle region may be provided to form the deformable region, while a thicker edge region may be provided to form the needle puncture region.

In this embodiment, the flexible sealing membrane 3 is a silicone membrane. However, the present disclosure is not limited thereto. In other embodiments, the flexible sealing membrane may be made of natural rubber, silicone rubber, or another biocompatible elastic flexible material.

This embodiment is described using an example in which the reservoir housing 1 and the flexible sealing membrane 3 are made of different materials. However, the present disclosure is not limited thereto. In other embodiments, as shown in FIG. 8, both the reservoir housing 1 and the flexible sealing membrane 3 may be made of a flexible biocompatible material, and the flexible sealing membrane 3 may be integrally formed with the reservoir housing 1 or bonded to the reservoir housing 1 to form a flexible upper portion of the reservoir. The flexible reservoir housing 1 facilitates pressing the chamber 11 to flush the ventricular catheter using the fluid in the chamber 11. In this case, the flexible sealing membrane 3 still provides a function of allowing cerebrospinal fluid withdrawal or therapeutic agent injection. A wall thickness at the flexible sealing membrane 3 may be substantially the same as a wall thickness of the reservoir housing 1. Alternatively, the wall thickness at the flexible sealing membrane 3 may be greater than the wall thickness of the reservoir housing 1 to increase the number of needle punctures and extend the service life of the reservoir. In this embodiment, both the reservoir housing 1 and the flexible sealing membrane 3 are made of silicone. However, the present disclosure is not limited thereto. In other embodiments, both may be made of natural rubber, silicone rubber, or another biocompatible flexible material.

In this embodiment, the sensor body 41 is disposed on an inner wall of the reservoir housing 1 or the chamber 11, specifically on an inner bottom wall of the reservoir housing 1 or the chamber 11. To avoid damage to the sensor body 41 caused by excessive force when a needle punctures the flexible sealing membrane 3 into the chamber 11, the reservoir housing 1 further has a protective portion extending to a position below the flexible sealing membrane 3 and overlying the sensor body 41. The protective portion is configured to block further advancement of a needle toward the sensor body 41 after the needle has pierced through the flexible sealing membrane 3, thereby protecting the sensor body 41 from being damaged by the needle. However, the present disclosure is not limited thereto. In other embodiments, the sensor body may be disposed on the inner bottom wall of the reservoir housing outside a projected area of the flexible sealing membrane, or the sensor body may be disposed on an inner side wall of the reservoir housing or the chamber 11.

Further, to prevent a needle from puncturing a bottom portion of the reservoir housing 1 that is made of a biocompatible polymer, in this embodiment the reservoir with wireless pressure monitoring further includes a puncture-resistant portion 6 disposed on an inner bottom surface of the reservoir housing 1 or inner bottom surface of the chamber 11. The puncture-resistant portion 6 is configured to block the needle so as to prevent the needle from damaging or penetrating the bottom portion of the reservoir housing 1 or the chamber 11. Specifically, the puncture-resistant portion 6 may be made of a biocompatible metal material, such as orthopedic stainless steel, a nickel-titanium alloy, or another metal. In this case, the metal puncture-resistant portion 6 may also serve as a part of the antenna to simplify the antenna structure. However, the present disclosure is not limited thereto. In other embodiments, the puncture-resistant portion 6 may also be made of a biocompatible polymer having relatively high hardness, such as polypropylene.

By positioning the sensor body 41 within the reservoir housing 1 or in the chamber 11 such that a sensing surface of the sensor body 41 is exposed to the reservoir chamber 11, the structure of the wireless pressure sensor 4 can be simplified and its sensing performance can be improved. In addition, the wireless pressure sensor 4 and the antenna 42 can be positioned closer to the skin surface of the patient, thereby reducing electromagnetic wave attenuation and improving wireless signal transmission. Further, in this configuration, the reservoir housing 1 itself may form a part of the antenna of the wireless pressure sensor 4, or an antenna structure may be formed on a bottom portion of the reservoir housing 1.

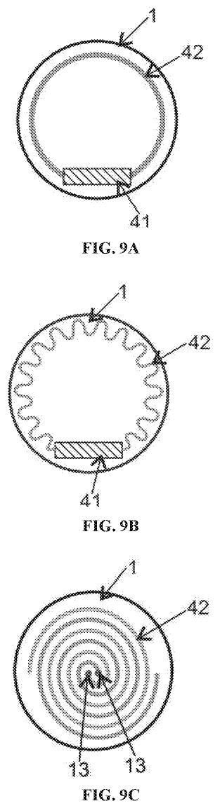

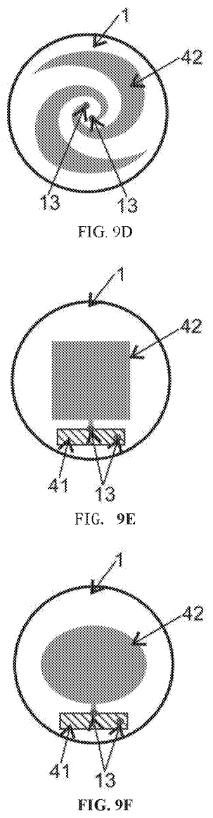

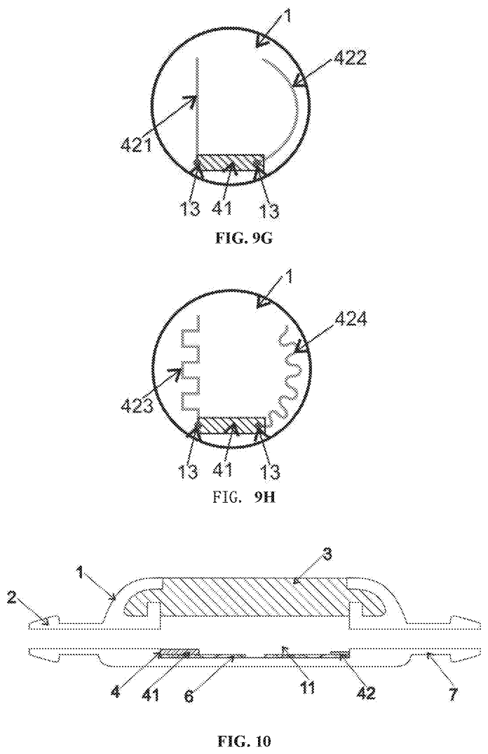

In this embodiment, the antenna 42 is disposed on a portion of the reservoir housing 1 where the sensor body 41 is located. Specifically, this portion of the reservoir housing 1 is made of a non-metallic biocompatible polymer or a biocompatible ceramic, and the antenna 42 is disposed on an inner surface of this portion, such as an inner bottom surface and/or an inner side wall. However, the present disclosure is not limited thereto. In other embodiments, the antenna may be disposed on an inner surface and/or an outer surface of the reservoir housing at the location corresponding to the sensor body, may be disposed on the outer surface of the reservoir housing, or may be embedded in the reservoir housing at the location corresponding to the sensor body. Alternatively, the reservoir housing may form part of the antenna. In other embodiments, when the reservoir housing includes a puncture-resistant portion, the sensor body may be disposed on the puncture-resistant portion, and the antenna may be disposed on the puncture-resistant portion and/or on the reservoir housing. The antenna may have any suitable configuration, including any one of the configurations shown in FIGS. 9A to 9H.

As shown in FIG. 9A, in this embodiment the antenna 42 is a circular ring metal antenna formed on the inner bottom surface of the reservoir housing 1 (or inner bottom surface of the chamber 11) or on the puncture-resistant portion in the chamber 11. Typically, the bottom of the reservoir housing 1 is a circular disk having a diameter between 15 mm and 25 mm, and the material of the reservoir housing 1 is a biocompatible polymer, such as polyether ether ketone (PEEK), polyaryletherketone (PAEK), or polytetrafluoroethylene (PTFE), or a biocompatible ceramic, such as aluminum oxide or zirconium oxide. The ring antenna or bent ring antenna shown in FIGS. 9A and 9B is formed at the bottom of the reservoir housing 1. The shape of the antenna matches the size and shape of the bottom of the reservoir housing 1 and can efficiently use available space in the reservoir. For example, the ring antenna or wavy annular antenna may be formed by embedding a metal wire into the inner bottom wall of the reservoir housing 1 or by electroplating on the inner bottom surface of the reservoir housing 1.

For the ring antenna of FIG. 9A, the circumference may be estimated using the following formula:

C=n·λeff

where C is the circumference of the ring antenna; n is a resonance coefficient selected according to a resonance mode; λeff is the effective wavelength in a medium and may be expressed as λeff=λ0/√εeff; λ0 is the free-space wavelength; and εeff is the effective dielectric constant, which may be estimated using a weighted average of a dielectric constant of cerebrospinal fluid and a dielectric constant of a material at the bottom of the reservoir housing 1.

In this embodiment, the operating frequency f of the antenna 42 is selected as 915 MHz or 865 MHz. With respect to the effective dielectric constant, because the upper surface of the antenna 42 contacts cerebrospinal fluid, the dielectric constant of cerebrospinal fluid in this frequency band is about 68. The lower surface of the antenna 42 contacts the inner bottom surface of the reservoir housing 1. When the reservoir housing 1 is a biocompatible polymer, the dielectric constant at the lower surface of the antenna 42 is about 3, and when the reservoir housing 1 is a ceramic, the dielectric constant at the lower surface of the antenna 42 is about 10. Therefore, a weighted-average approximation may be used to estimate the effective dielectric constant. Based on the above formula, when the operating frequency is 915 MHz, the antenna diameter is about 17 mm, and when the operating frequency is 865 MHz, the antenna diameter is about 18 mm. In this case, the outer diameter of the antenna substantially corresponds to the diameter of the inner bottom surface of the reservoir housing 1, which may be 15 mm to 25 mm. Further, for a small-size reservoir used for children, a bent ring antenna (FIG. 9B) may be used to tune the antenna 42 to the corresponding operating frequency.

However, the present disclosure is not limited thereto. In other embodiments, the antenna may have a wavy annular shape, as shown in FIG. 9B; a spiral shape with a uniform line width, as shown in FIG. 9C; a spiral shape with a non-uniform line width, as shown in FIG. 9D; a polygonal patch shape, as shown in FIG. 9E; or a circular patch shape, as shown in FIG. 9F. The antenna may also include one or more of a straight line, a bent line, or an arcuate line. For example, in FIG. 9G, the antenna 42 includes a straight line segment 421 and an arcuate line segment 422. In FIG. 9H, the antenna 42 includes a meandered line segment 423 and a wavy arcuate line segment 424. A more complex antenna structure may improve antenna radiation efficiency, but may also increase manufacturing complexity. FIGS. 9C to 9H show antenna structures in which the antenna 42 is disposed on an outer surface of the reservoir housing 1 at a location corresponding to the sensor body 41 and/or embedded in the reservoir housing 1 at the location corresponding to the sensor body 41. In this case, the reservoir housing 1 includes hermetic electrical feedthroughs 13, and opposite ends of the antenna 42 are electrically coupled to the sensor body 41 through the hermetic electrical feedthroughs 13. FIGS. 9C and 9D show antenna structures on the outer surface of the reservoir housing 1. Due to the viewing angle, the sensor body 41 is not shown in FIGS. 9C and 9D.

Although this embodiment is described using an example in which the reservoir housing 1 is made of a non-metallic biocompatible polymer or a biocompatible ceramic, the present disclosure is not limited thereto. In other embodiments, a portion of the reservoir housing at which the sensor body is located may be made of a biocompatible metal material. In this case, the metal portion of the reservoir housing may form part of the antenna, thereby further simplifying the antenna arrangement.

In this embodiment, the reservoir housing 1 is an integral structure. However, the present disclosure is not limited thereto. In other embodiments, the reservoir housing may also be a separate structure, including an upper housing and a lower housing that are sealingly connected, and the upper housing and the lower housing may be connected by bonding or welding.

The reservoir with wireless pressure monitoring provided in this embodiment may be implanted in patients requiring monitoring and management of ICP, such as patients with acute traumatic brain injury, brain tumors, or cerebral hemorrhage. Exemplary implantation applications of the reservoir with wireless pressure monitoring are described below. However, the present disclosure is not limited thereto, and the reservoir with wireless pressure monitoring may be implanted in any patient requiring monitoring and management of ICP.

For a patient with acute traumatic brain injury, after the reservoir with wireless pressure monitoring is implanted, the scalp may be sutured so that the implantation site is closed, thereby interrupting a transcutaneous infection pathway. ICP can be monitored wirelessly, thereby providing a monitoring function similar to that of an implantable ICP monitoring approach. When the ICP of the patient is elevated, a needle may be passed through the scalp and the flexible sealing membrane 3 to withdraw cerebrospinal fluid, thereby reducing ICP. According to clinical needs and ICP data provided by the reservoir with wireless pressure monitoring, cerebrospinal fluid may be withdrawn multiple times. Alternatively, the needle may be fixed by an additional device to continuously drain cerebrospinal fluid, thereby providing ICP monitoring and cerebrospinal fluid drainage. The reservoir with wireless pressure monitoring provided by the present disclosure can reduce infection risk while enabling ICP monitoring and cerebrospinal fluid drainage for controlling ICP. In addition, longer-term monitoring in a hospital ward can be achieved. The wireless function facilitates free movement of the patient and reduces the risk that a patient, particularly a child, pulls out or damages a lead of a wired monitoring device.

For a patient with a brain tumor or cerebral hemorrhage, after craniotomy, the reservoir with wireless pressure monitoring may be implanted and the scalp may be sutured. ICP monitoring may be used to determine whether elevated ICP, bleeding, infection, or other postoperative complications are present, and may guide cerebrospinal fluid withdrawal to reduce ICP. A needle may also be passed through the scalp and the flexible sealing membrane 3 to withdraw cerebrospinal fluid for laboratory analysis to assess bleeding or infection. A therapeutic agent may be injected through the flexible sealing membrane 3 to provide treatment. The reservoir with wireless pressure monitoring is suitable for long-term implantation and may be retained in the patient after discharge from the hospital, so that ICP can be periodically monitored, cerebrospinal fluid can be withdrawn to reduce ICP, laboratory analysis and therapeutic agent administration can be performed, and the ventricular catheter 5 can be flushed by pressing the flexible sealing membrane 3. The reservoir may be used for postoperative monitoring, rehabilitation, and care of patients with brain tumors or cerebral hemorrhage.

In some embodiments, as shown in FIG. 10, the ventricular catheter connector 2 is disposed on a side wall of the reservoir housing 1, and is in fluid communication with the chamber 11, and the reservoir with wireless pressure monitoring further includes a peritoneal/atrial shunt catheter connector 7 disposed on another side wall of the reservoir housing 1 opposite the ventricular catheter connector 2 and in fluid communication with the reservoir chamber 11. However, the present disclosure is not limited thereto. In other embodiments, as shown in FIG. 13, the ventricular catheter connector 2 is disposed at a bottom wall of the reservoir housing 1, and the peritoneal/atrial shunt catheter connector 7 is disposed on a side wall of the reservoir housing 1. Two connectors 2 and 7 are in fluid communication with the chamber 11.

Further, as shown in FIG. 11, in this embodiment, a peritoneal/atrial shunt catheter 8 is detachably connected to the peritoneal/atrial shunt catheter connector 7. The peritoneal/atrial shunt catheter connector 7 and the peritoneal/atrial shunt catheter 8 are configured to drain cerebrospinal fluid from the reservoir chamber 11 to a peritoneal cavity or an atrium of a patient. Further, the peritoneal/atrial shunt catheter 8 may include a flow control valve having an adjustable opening, and the wireless pressure sensor 4 may provide pressure information for adjustment of the flow control valve. For example, when the ICP is elevated, a clinician may increase the opening of the flow control valve to increase a shunt flow rate. When the ICP is low, the clinician may reduce the opening of, or close, the flow control valve to reduce or stop cerebrospinal fluid drainage. Accordingly, the reservoir is suitable for a patient requiring implantation of a peritoneal/atrial shunt catheter.

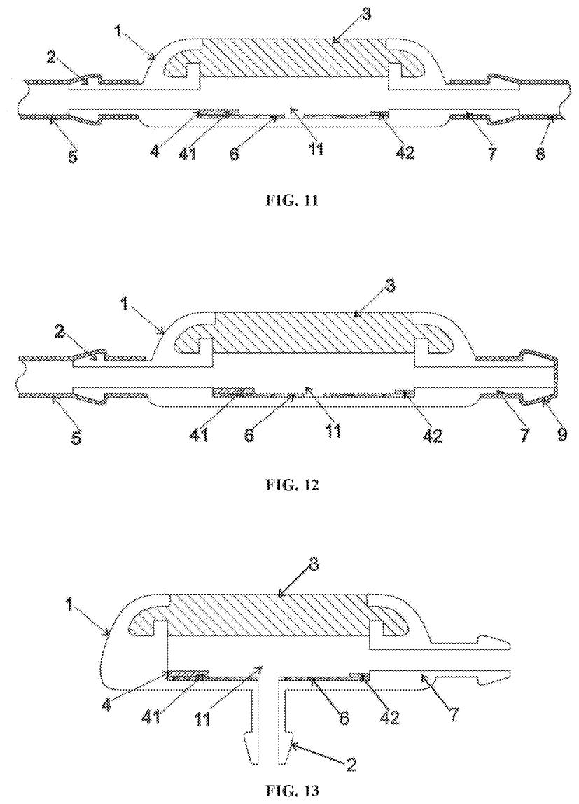

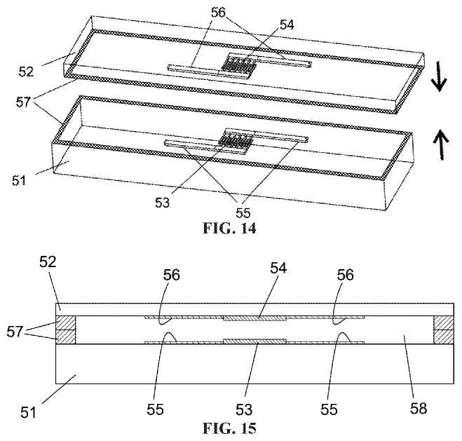

For a patient for whom it is uncertain whether a ventriculoperitoneal or ventriculoatrial shunt catheter is required, or for whom implantation of such a shunt catheter is not yet clinically appropriate while ICP still needs to be monitored, this embodiment provides a configuration suitable for such clinical use. As shown in FIG. 12, the reservoir with wireless pressure monitoring further includes a removable sealing cap 9 detachably disposed on the peritoneal/atrial shunt catheter connector 7. The detachable arrangement of the peritoneal/atrial shunt catheter 8 and the removable sealing cap 9 allows the two components to be selectively used or replaced during different treatment stages of the patient. Specifically, at an early stage, the reservoir with wireless pressure monitoring having the removable sealing cap 9 may be implanted to provide ICP monitoring, flushing of the ventricular catheter 5, therapeutic agent administration, and cerebrospinal fluid withdrawal and laboratory analysis. When the patient requires implantation of the peritoneal/atrial shunt catheter 8, surgery may be performed to remove the removable sealing cap 9 and install the peritoneal/atrial shunt catheter 8, thereby draining cerebrospinal fluid to the peritoneal cavity or atrium to reduce ICP, as shown in FIG. 11.

Specifically, for a patient with congenital hydrocephalus, normal-pressure hydrocephalus, or a patient requiring implantation of a ventriculoperitoneal or ventriculoatrial shunt catheter, when the patient has an intracranial infection, hemorrhage, or another condition such that implantation of the shunt catheter is not yet clinically appropriate, a conventional reservoir may be implanted. A therapeutic agent may be administered through the conventional reservoir to treat or control the infection or other condition, and ICP may be controlled by withdrawing cerebrospinal fluid. However, because the conventional reservoir does not provide real-time ICP data, it may not provide sufficient guidance regarding the timing or amount of cerebrospinal fluid withdrawal.

The reservoir with wireless pressure monitoring provided by the present disclosure can provide real-time ICP data of the patient, thereby providing guidance regarding the timing and amount of cerebrospinal fluid withdrawal. The reservoir can also be used to flush the ventricular catheter 5 to reduce or prevent blockage. When implantation of the ventriculoperitoneal or ventriculoatrial shunt catheter becomes clinically appropriate, the removable sealing cap 9 may be removed, and the peritoneal/atrial shunt catheter 8 may be directly connected to the peritoneal/atrial shunt catheter connector 7 to shunt cerebrospinal fluid to the peritoneal cavity or atrium.

In addition, when the peritoneal/atrial shunt catheter connector 7 is connected to the peritoneal/atrial shunt catheter 8, the reservoir with wireless pressure monitoring may further include a valve disposed on/in the peritoneal/atrial shunt catheter connector 7 or on/in the peritoneal/atrial shunt catheter 8. When the flexible sealing membrane 3 is pressed to incease the pressure in the chamber 11 to make the cerebrospinal fluid flush the ventricular catheter 5, the valve disposed in a flow path leading to the peritoneal/atrial shunt catheter 8 automatically closes, so that cerebrospinal fluid in the reservoir chamber 11 flows toward the ventricular catheter 5, thereby improving the flushing effect.

Although this embodiment is described using patients with congenital hydrocephalus, normal-pressure hydrocephalus, or patients requiring implantation of a ventriculoperitoneal or ventriculoatrial shunt catheter as examples, the present disclosure is not limited thereto. The reservoir with wireless pressure monitoring provided in this embodiment is also applicable to other patients requiring monitoring and management of ICP, such as patients with acute traumatic brain injury, brain tumors, or cerebral hemorrhage as described in some embodiments as well as patients undergoing lumboperitoneal shunting.

The reservoir housing 1, the ventricular catheter connector 2, and the peritoneal/atrial shunt catheter connector 7 may be made of the same material or different materials, and each may be made of a biocompatible metal or a biocompatible polymer. When the same material is used, the reservoir housing 1, the ventricular catheter connector 2, and the peritoneal/atrial shunt catheter connector 7 may be integrally formed or may be connected by bonding or welding.

In some embodiments, specific structures and possible configurations of the reservoir housing 1, the ventricular catheter connector 2, the flexible sealing membrane 3, and the sensor body 41 and the antenna 42 of the wireless pressure sensor 4 are the same as those described in the aforementioned embodiments.

Further, because the reservoir is a long-term implantable device, in some embodiments, the sensor body 41 uses quartz-to-quartz bonding to encapsulate the SAW resonator or FBAR therein, so as to improve long-term hermetic sealing of the device. A SAW resonator is described below as an example. As shown in FIG. 14, a first SAW resonator 54 is fabricated on a quartz wafer 52, and the quartz wafer 52 is thinned to less than 100 μm.

To reduce or compensate for pressure sensor drift caused by environmental temperature changes, aging, or other factors in the environment of the SAW resonator, the sensor body 41 may further include a second SAW resonator 53. The second SAW resonator 53 is formed on a second, thicker quartz wafer 51, which generally has a thickness greater than 200 μm. The two quartz wafers have the same cut angle and material properties. Accordingly, environmental temperature changes, aging, or other common-mode factors affect the resonant frequencies of the two SAW resonators in a similar manner, so that a difference between the two resonant frequencies, for example 2 MHz, can be used to reduce or compensate for drift caused by such factors. However, the present disclosure is not limited thereto. In other embodiments, the sensor body 41 may include only one SAW resonator.

With respect to the encapsulation structure of the SAW resonator, in this embodiment, the package is formed by bonding the quartz wafer 52, a quartz wafer 57 having a cavity, and the second quartz wafer 51. However, the present disclosure is not limited thereto. In other embodiments, the package may be formed by bonding the quartz wafer 52, an intermediate layer having a cavity formed in glass or metal, and the second quartz wafer 51. After encapsulation, bonding regions surrounding the two SAW resonators form a hermetic cavity 58. Electrical leads 55 and 56 at two ends of each SAW resonator are connected to the antenna 42 of the wireless pressure sensor through the bonding regions or the wafers. However, the present disclosure is not limited thereto. In other embodiments, the electrical leads may be routed to an edge or a bottom of the pressure sensor through through-wafer vias (TWVs), through-glass vias (TGVs), or a redistribution layer (RDL), and then electrically connected to antenna terminals of the pressure sensor.

The specific working principle is as follows. An external reader emits radio-frequency electromagnetic waves. The electromagnetic waves are coupled through an external antenna to the antenna 42 of the wireless pressure sensor, thereby exciting the SAW resonators 53 and 54 in the sensor body 41. When pressure in the reservoir chamber 11 changes, the quartz wafer 52 deforms, thereby changing the resonant frequency of the SAW resonator 54. An echo signal from the resonator is returned to the external reader through the antenna 42 of the wireless pressure sensor and the external antenna. The external reader determines the pressure in the reservoir chamber 11 by detecting a frequency shift of the echo signal returned by the resonator.

In summary, after the reservoir with wireless pressure monitoring provided by the present disclosure is implanted under the scalp of a patient, the scalp may be sutured so that the implantation site is closed, thereby interrupting a transcutaneous infection pathway and enabling long-term monitoring. The wireless monitoring function facilitates free movement of the patient and reduces the risk that a patient, particularly a child, pulls out or damages a lead of a wired monitoring device. Further, the small-volume wireless pressure sensor supports a miniaturized design of the reservoir, so that the reservoir can be suitably implanted under the scalp of the patient. Alternatively, when the size of the reservoir is substantially the same as that of a conventional reservoir, the miniaturized wireless pressure sensor enables the reservoir chamber to have a larger volume. In this case, when the flexible sealing membrane is pressed, a greater amount of cerebrospinal fluid is driven from the reservoir chamber into the ventricular catheter, thereby enhancing the effect of flushing the ventricular catheter.

In addition, symptoms associated with blockage of a ventricular catheter, such as headache, urinary dysfunction, and abnormal gait, may be similar to symptoms caused by other conditions. Existing reservoirs generally do not provide pressure information for determining whether a ventricular catheter is blocked. The reservoir with wireless pressure monitoring provided by the present disclosure can assist in determining whether the ventricular catheter is blocked based on ICP data, thereby helping to avoid unnecessary catheter replacement surgery and conserve medical resources.

The present disclosure improves an existing reservoir by adding additional functions while retaining therapeutic agent administration and cerebrospinal fluid withdrawal functions.
1. A wireless pressure sensor is integrated into the reservoir to provide ICP monitoring. After the reservoir is implanted, the scalp may be sutured so that the implantation site is closed, thereby reducing the risk of transcutaneous infection associated with conventional ICP monitoring devices and cerebrospinal fluid catheters.
2. When implantation of a cerebrospinal fluid shunt catheter is not yet clinically appropriate, for example when intracranial hemorrhage, infection, or another condition is present, the reservoir may be used as a temporary cerebrospinal fluid drainage and therapeutic agent delivery device. The reservoir may also serve as an alternative to a cerebrospinal fluid catheter for certain drainage and delivery functions in a hospital or in an out-of-hospital setting. Pressure data provided by the reservoir can guide the timing and amount of cerebrospinal fluid drainage.
3. The reservoir may be implanted over an extended period and connected to a conventional cerebrospinal fluid shunt catheter to discharge excess cerebrospinal fluid to the peritoneal cavity or atrium.
4. By periodically pressing the reservoir, the ventricular catheter can be flushed, thereby reducing catheter blockage caused by blood clots, choroid plexus tissue, brain tumor tissue, cerebrospinal fluid deposits, or the like. A valve may be disposed on the peritoneal/atrial shunt catheter connector or on the peritoneal/atrial shunt catheter. When the flexible sealing membrane is pressed to flush the ventricular catheter, the valve disposed in a flow path leading to the peritoneal/atrial shunt catheter automatically closes, so that cerebrospinal fluid in the reservoir chamber flows toward the ventricular catheter to improve the flushing effect.
5. Pressure data output by the reservoir can be used to adjust a cerebrospinal fluid shunt catheter valve, thereby reducing excessive or insufficient cerebrospinal fluid drainage and helping to maintain ICP within a desired range.
6. The reservoir can assist in determining whether the ventricular catheter is blocked, thereby helping to avoid unnecessary catheter replacement surgery.

Although the present disclosure has been described above with reference to exemplary embodiments, the embodiments are not intended to limit the present disclosure. A person skilled in the art may make various changes and modifications without departing from the spirit and scope of the present disclosure. Therefore, the scope of protection of the present disclosure should be determined by the claims

1. A reservoir with wireless pressure monitoring, comprising:
a reservoir housing comprising a chamber configured for storing cerebrospinal fluid; and
an aspiration/injection window;
a ventricular catheter connector disposed on the reservoir housing and in fluid communication with the chamber;
a flexible membrane sealingly covering the aspiration/injection window; and
a wireless pressure sensor comprising a sensor body and an antenna, the wireless pressure sensor being disposed in the chamber; wherein the wireless pressure sensor is configured to sense a pressure in the chamber and to transmit a signal corresponding to the sensed pressure to an external receiver;
wherein the wireless pressure sensor is based on a surface acoustic wave (SAW) resonator or a thin-film bulk acoustic resonator (FBAR).

2. The reservoir with wireless pressure monitoring of claim 1, wherein the reservoir housing is an integral structure; or wherein the reservoir housing comprises an upper housing and a lower housing that are separately formed and sealingly connected to each other, the upper housing and the lower housing being connected by bonding or welding.

3. The reservoir with wireless pressure monitoring of claim 1, wherein the wireless pressure sensor has dimensions less than or equal to 10×3×1 mm³, or the reservoir housing comprises a circular cross-section having a maximum diameter of 15 mm to 25 mm, with a height of 5 mm to 10 mm.

4. The reservoir with wireless pressure monitoring of claim 1, further comprising a ventricular catheter, wherein one end of the ventricular catheter is connected to the ventricular catheter connector and another end of the ventricular catheter is configured to be implanted in a ventricle of a patient, wherein the ventricular catheter is separately connected to the ventricular catheter connector or is integrally connected therewith, and wherein the ventricular catheter connector is disposed on a bottom wall or a side wall of the reservoir housing.

5. The reservoir with wireless pressure monitoring of claim 1, wherein the flexible sealing membrane is a flexible elastic membrane made of natural rubber, silicone, or silicone rubber, and wherein, when the flexible sealing membrane is pressed by an external force so as to deform inwardly toward the chamber to increase a pressure in the chamber so that a cerebrospinal fluid in the ventricular catheter is forced through a drainage opening of the ventricular catheter.

6. The reservoir with wireless pressure monitoring of claim 1, wherein the flexible sealing membrane is a planar membrane or a curved membrane having a central portion protruding toward the scalp of a patient; wherein the flexible sealing membrane has a substantially uniform thickness; or wherein the flexible sealing membrane comprises a needle puncture region located in a central region of the sealing membrane and a deformable region located around the needle puncture region; wherein the needle puncture region has a thickness greater than a thickness of the deformable region.

7. The reservoir with wireless pressure monitoring of claim 1, wherein the reservoir housing is made of a biocompatible polymer, a biocompatible ceramic, or a biocompatible metal; wherein the antenna is disposed on an inner surface and/or an outer surface of the reservoir housing; or wherein the antenna is embedded in the reservoir housing; or wherein the antenna is disposed on the reservoir housing or the reservoir housing forms a part of the antenna.

8. The reservoir with wireless pressure monitoring of claim 1, further comprising a puncture-resistant portion disposed on an inner bottom surface of the reservoir housing, wherein the puncture-resistant portion is made of a biocompatible metal, a biocompatible ceramic, or a biocompatible polymer having a hardness greater than a hardness of the reservoir housing, so as to prevent a needle from damaging or penetrating a bottom portion of the reservoir housing, or wherein the antenna is disposed on the puncture-resistant portion or the puncture-resistant portion forms a part of the antenna.

9. The reservoir with wireless pressure monitoring of claim 1, wherein the sensor body is disposed on an inner bottom surface or a puncture-resistant portion of the reservoir housing and wherein the reservoir housing comprises a protective portion extending to a position below the flexible sealing membrane and overlying the sensor body, the protective portion being configured to block a needle advanced toward the sensor body after the needle has pierced through the flexible sealing membrane, thereby preventing the needle from damaging the sensor body.

10. The reservoir with wireless pressure monitoring of claim 7, wherein, when the antenna is disposed on an outer surface of the reservoir housing at a location corresponding to the sensor body or is embedded in the reservoir housing at the location corresponding to the sensor body, the reservoir housing has one or more wiring holes, and two ends of the antenna are electrically connected to the sensor body through the one or more wiring holes.

11. The reservoir with wireless pressure monitoring of claim 7, wherein the antenna is one of an annular antenna, a wavy annular antenna, a spiral antenna having a uniform line width, a spiral antenna having a non-uniform line width, a polygonal patch antenna, or a circular patch antenna; or wherein the antenna comprises one or more of a straight line, a bent line, or an arcuate line.

12. The reservoir with wireless pressure monitoring of claim 1, further comprising a peritoneal/atrial shunt catheter connector disposed on a side wall of the reservoir housing and in fluid communication with the chamber, wherein the peritoneal/atrial shunt catheter connector is detachably connectable to a removable sealing cap or to a peritoneal/atrial shunt catheter that is connected to a peritoneal cavity or an atrium of a patient.

13. The reservoir with wireless pressure monitoring of claim 12, further comprising a valve disposed on/in the peritoneal/atrial shunt catheter connector or on/in the peritoneal/atrial shunt catheter, wherein, when the flexible sealing membrane is pressed and increase the pressure in the chamber so that the increased pressure forces a cerebrospinal fluid to flush a ventricular catheter, the valve automatically closes so that the cerebrospinal fluid in the chamber is directed toward the ventricular catheter, thereby improving a flushing effect and reducing a risk that excessive pressure is applied to a ventricle or brain tissue.

14. The reservoir with wireless pressure monitoring of claim 13, wherein the reservoir housing, the ventricular catheter connector, and the peritoneal/atrial shunt catheter connector are made of the same material or different materials, each being selected from a biocompatible metal, a biocompatible polymer, or a biocompatible ceramic.

15. The reservoir with wireless pressure monitoring of claim 1, wherein at least a portion of the reservoir housing is made of a flexible biocompatible material, wherein the flexible sealing membrane is made of the flexible biocompatible material, and wherein the flexible sealing membrane is integrally formed with the reservoir housing or bonded thereto to form a flexible upper portion of the reservoir.
